(19) United States
(12) Patent Application Publication
(10) Pub. No.: US 2008/0206134 A1
(43) Pub. Date: Aug. 28, 2008

(54) Radio-opaque hemostatic agents and devices and methods for the delivery thereof

(76) Inventors: Denny Lo, Bethlehem, CT (US); Giacomo Basadonna, Haddam, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 11/710,106

(22) Filed: Feb. 22, 2007

(51) Int. Cl. A61K 51/02; A61K 101/00; A61M 36/04

(52) U.S. Cl. 424/1.61; 424/9.31

(57) ABSTRACT

A radiographic composition for promoting the formation of clots in blood includes a zeolite and iodine. A device for promoting the clotting of blood at an internal wound site includes a catheter; a delivery instrument insertable through the catheter; a radioopaque blood clotting agent that deliverable through the delivery instrument; and a positive pressure apparatus that can dispense the blood clotting agent to the wound site through the delivery instrument. A system for radiographically imaging an internally bleeding wound includes means for delivering a radioopaque blood clotting agent and means for radiographically imaging the radioopaque blood clotting agent. A method of imaging an internally bleeding wound includes the steps of inserting a catheter into a patient; advancing the catheter to a point adjacent the bleeding wound; depositing a radioopaque zeolite at the bleeding wound; and imaging the radioopaque zeolite to monitor blood-clotting caused by the zeolite.

### TECHNICAL FIELD

The present invention relates generally to blood clotting agents and, more particularly, to blood clotting agents having radio-opacity and devices incorporating such agents as materials for use in monitoring and controlling blood flow.

### BACKGROUND OF THE INVENTION

Various techniques can be utilized to view the internal body structures of humans and animals. Such techniques include magnetic resonance imaging (MRI) and radiography. In MRI techniques, pharmaceutical compositions comprising paramagnetic ions are captured within zeolite structures either as “free” ions within zeolite cages or as complexes with appropriate complexing agents. The free ions are typically charged species that lack ligands, although interactions between species are also possible. Such interactions may be in the form of counterion interactions within the structures of the capturing zeolite or between captured ions and the structure of the capturing zeolite itself. The paramagnetic ions are typically ions derived from complexes of gadolinium (viz., diethylenetriamine penta-acetic acid complexes of trivalent gadolinium), which are incorporated into the zeolite using ion-exchange methods. The gadolinium/zeolite is then orally or intravenously administered to a patient and imaged.

Radiographic methods involve the introduction of a radiographic contrast agent into tissue and the subsequent tracking of the agent using x-ray imaging techniques. One type of radiographic contrast agent is an aqueous suspension of barium sulfate, which is swallowed by a patient or administered as an enema. The contrast agent is then imaged to determine its movement through the digestive tract. Another type of radiographic contrast agent is based on iodine bound in a non-ionic or in an ionic compound, which may be administered intravenously, intraarterially, intrathecally, or intraabdominally. Typical non-ionic compounds include iodipamide and iohexyl, although other compounds based on (2,3-dihydroxypropyl)isophthalamides may also be utilized. In intravenous or intraarterial applications, the iodine-based compounds may be imaged and used to map the flow of blood.

Blood is a liquid tissue that includes red cells, white cells, corpuscles, and platelets dispersed in a liquid phase. The liquid phase is plasma, which includes acids, lipids, solubilized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

Controlling the flow of blood from compromised tissue is of concern when either traumatic injury is sustained or when surgical procedures are undertaken. The location of the bleed site can make controlling bleeding easier; on the other hand, the location can sometimes make controlling the bleeding considerably more difficult. When the blood is emanating from an indiscernible internal location and is pooling within adjacent tissue and accumulating around organs, the bleed site can be especially difficult to locate and treat.

Once the location of the bleed site is discerned, the site must be closed to prevent further blood loss. Sponges or balloon-type devices can be used to exert pressure against the bleed site and to absorb blood. More invasive procedures such as suturing or stapling can also be used, although some types of tissue typically do not respond favorably to such techniques. In situations in which sponging, blocking, suturing, or stapling are less than adequate, seepage can occur, which may result in excessive and unnoticed blood loss.

The available methods of monitoring the flow of blood from internal bleed sites, as indicated above, as well as available methods of stopping blood flow all have drawbacks and limitations. There is a need for improved systems, methods, and devices that can quickly monitor and address the bleeding or hemorrhaging associated with internal wounds. Regarding procedures and devices for monitoring and stopping the bleeding, the need for a quick, effective imaging and clotting technique has been long recognized in both human and veterinary surgery.

Based on the foregoing, it is a general object of the present invention to provide compositions, devices, and systems for imaging and controlling internal bleeding and methods of their use that overcome the problems with or improve upon the prior art.

### SUMMARY OF THE INVENTION

According to one aspect, the present invention resides in a radiographic composition for promoting the formation of clots in blood. Such a composition comprises a zeolite and an associated compound having an effective amount of iodine present in ionic form. The iodine, which may be from iodipamide, iohexyl, or the like, provides a radioopaque quality to the composition (i.e., the zeolite/iodine composition can be radiographically imaged using x-rays). Prior to being used in a patient, a moisture content of zeolite/iodine composition is adjusted by drying, re-hydrating, or a combination of drying and re-hydrating. Adjusting the moisture enables the composition to have a specific moisture content, which enables the exothermic effects of the composition in blood to be controlled, thereby preventing the composition from becoming so hot as to cause discomfort in the patient during use.

In a second aspect, the present invention resides in a device for promoting the clotting of blood at an internal wound site. The device comprises a catheter; a delivery instrument that can be inserted through the catheter; a radioopaque blood clotting agent that can be delivered through the delivery instrument; and a positive pressure apparatus such as a pump or syringe that can dispense the blood clotting agent to the wound site through the delivery instrument. The radioopaque blood clotting agent comprises a molecular sieve material (preferably a zeolite) and preferably an iodine compound. Depositing the radioopaque blood clotting agent at the bleeding wound site causes the molecular sieve material to come into contact with blood, which clots the blood. The radioopacity of the blood clotting agent allows it to be imaged as the clotting is taking effect.

In a third aspect, the present invention resides in a system for radiographically imaging an internally bleeding wound. Such a system comprises means for delivering a radioopaque blood clotting agent to the internally bleeding wound and means for radiographically imaging the radioopaque blood clotting agent. Means for delivering the blood clotting agent to the internally bleeding wound may be a conduit, and the blood clotting agent may be delivered through the conduit using a positive pressure device such as a pump or a syringe. Means for radiographically imaging the blood clotting agent may be an x-ray apparatus.

In a fourth aspect, the present invention resides in a method of imaging an internally bleeding wound. The method includes the steps of inserting a catheter into a patient; advancing the catheter to a point adjacent the bleeding wound; depositing a radioopaque zeolite from a delivery instrument in the catheter at the bleeding wound; imaging the radioopaque zeolite to monitor blood-clotting caused by the zeolite. The radioopaque zeolite is imaged using x-rays.

In the preferred embodiments of the present invention, the zeolite is found to be particularly effective in causing blood to clot. The compositions, devices, methods, and systems of the present invention are especially useful in controlling bleeding. One advantage of the present invention is that upon use of any of the compositions or devices of the present invention to address the flow of blood from a blood vessel or an internal organ, the clotting effect can be monitored radiographically.

Another advantage of the present invention is that the devices described herein can be easily retracted from the patient with little adverse effect. In particular, the devices can be easily and cleanly pulled away from the wound while leaving only the zeolite necessary for clotting remaining at the bleed site.

Another advantage is that the small particle size of the zeolite material produces a less drastic exothermic reaction than does a larger particle size. The porous nature of the zeolite still allows liquid blood constituents to be wicked away to cause thickening of the blood, thereby facilitating the formation of clots. Because the initial moisture content of the zeolite is controlled in a preferred embodiment, a less aggressive drawing of moisture from the blood can be realized, which thereby tempers any exothermic effects due to the absorption of water by the zeolite experienced at the wound site.

### BRIEF DESCRIPTION OF THE DRAWINGS

FIG. 1 is a perspective view of a blood clotting device, of the present invention, capable of dispensing a blood clotting agent.

FIG. 2 is a sectional view of one exemplary embodiment of a conduit for use with the device of FIG. 1, the conduit having a wax coating to prevent the absorption of aqueous body fluids by the blood clotting agent.

FIG. 3 is a sectional view of another exemplary embodiment of a conduit for use with the device of FIG. 1, the conduit having a frangible membrane to prevent the absorption of aqueous body fluids by the blood clotting agent.

FIG. 4 is a schematic representation of a system, of the present invention, for imaging radiographic zeolite.

FIG. 5 is a schematic representation of a system, of the present invention, utilizing a plurality of apparatuses that image radiographic zeolite to determine parameters of an internal wound.

### DETAILED DESCRIPTION OF THE PREFERRED EMBODIMENTS

Disclosed herein are devices, methods, and systems for delivering radioopaque hemostatic materials to internal wounds to promote the clotting of blood, thereby providing for the cessation of blood flow from the wound. The devices can be inserted subcutaneously through ports during invasive and minimally invasive surgical procedures to supply the radioopaque hemostatic (blood clotting) agents to bleeding wound sites. By contacting the blood with the blood clotting agent at the wound site, at least portions of the liquid phases of the blood are absorbed, thereby promoting clotting. The radioopaque attributes of the blood clotting agents allow the blood flow to be radiographically monitored before, during, and after clotting takes effect. The blood clotting agent is preferably a particulate molecular sieve material having an associated radioopaque component that can be maintained in direct contact with blood emanating from a wound.

The molecular sieve material used in the present invention may be a synthetic polymer gel, cellulose material, porous silica gel, porous glass, alumina, hydroxyapatite, calcium silicate, zirconia, zeolite, or the like. Exemplary synthetic polymers include, but are not limited to, stylene-divinylbenzene copolymer, cross-linked polyvinyl alcohol, cross-linked polyacrylate, cross-linked vinyl ether-maleic anhydride copolymer, cross-linked stylene-maleic anhydride copolymer or cross-linked polyamide, and combinations thereof.

The molecular sieve material is preferably a zeolite. Other molecular sieve materials that may be used include, but are not limited to, faujasite. As used herein, the term “zeolite” refers to a crystalline form of aluminosilicate having the ability to be dehydrated without experiencing significant changes in the crystalline structure. The zeolite may include one or more ionic species such as, for example, calcium and sodium moieties. Typically, the preferred zeolite is a powdered, friable material that is less than about 75% by weight silicon oxide, and preferably less than about 65% by weight silicon oxide; less than about 50% by weight aluminum oxide, and preferably less than about 40% by weight aluminum oxide; less than about 30% by weight sodium oxide, and preferably less than about 20% by weight of sodium oxide; less than about 30% by weight of calcium oxide, and preferably less than about 20% by weight of calcium oxide. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings. The median size of the zeolite particle used is about 7 microns. However, the present invention is not limited in this regard as other sizes of zeolite particle are within the scope of the invention.

The zeolite may be mixed with or otherwise used in conjunction with other materials having the ability to be dehydrated without significant changes in crystalline structure. Such materials include, but are not limited to, magnesium sulfate, sodium metaphosphate, calcium chloride, dextrin, polysaccharides, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, anal cite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides. One zeolite material found to be particularly useful in practicing the present invention is Molsiv Adsorbents 5A, manufactured by UOP LLC of Des Plaines, Ill. However, the present invention is not limited in this regard as other zeolite materials can be substituted without departing from the broader aspects of the present invention.

The radioopacity of the zeolite is derived from the association of an iodine-based compound with the zeolite structure. The association may be either by binding the iodine-based compound to the zeolite using a binding agent, by blending the compound with the zeolite, or by incorporating the compound into the crystalline structure of the zeolite. Iodine compounds suitable for use with the zeolite include, but are not limited to, iodipamide, iohexyl, compounds based on 5-amino-2,4,6-triiodo-N,N′-bis(2,3-dihydroxypropyl)isophthalamide as building blocks, compounds based on 5-acetylamino-2,4,6-triiodo-N,N′-bis(2,3-dihydroxypropyl)isophthalamide as building blocks, combinations of the foregoing, and the like. The present invention is not limited in this regard, however, as other iodine-based compounds are within the scope of the present invention.

Various other materials may be mixed with, associated with, or incorporated into the zeolite/iodine compounds to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, antimicrobial agents, anti-inflammatory agents, analgesics (e.g., cimetidine, chloropheniramine maleate, diphenhydramine hydrochloride, and promethazine hydrochloride), bacteriostatics, compounds containing silver ions, and the like. Still other materials that can be incorporated to provide additional hemostatic functions include ascorbic acid, tranexamic acid, rutin, and thrombin. Botanical agents having desirable effects on the wound site may also be added.

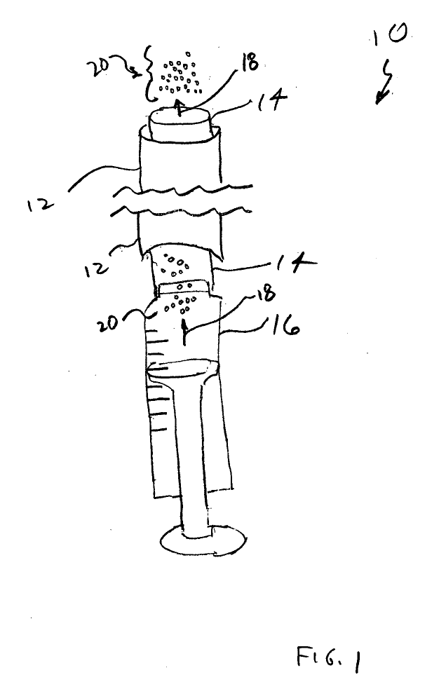

In one embodiment of the present invention shown in FIG. 1, a device that can be used to facilitate the clotting of blood at an internal bleed site is shown at reference numeral 10 and is hereinafter referred to as “device 10.” The device 10 is especially useful for delivering the radioopaque zeolites of the present invention to a wound to close the wound to prevent undesirable internal bleeding and the formation of hematoma (pooling of blood). The wound can be located using any suitable method. The device 10 comprises a catheter 12, at least one delivery instrument such as a conduit 14 through which the radioopaque zeolite may be dispensed, and a positive pressure apparatus 16. The conduit 14 is configured to be advanced through the catheter 12 or with the catheter to the wound site. The positive pressure apparatus 16 may be a syringe or the like, although the present invention is not limited in this regard as other devices (e.g., pumps, blowers, and the like) are within the scope of the invention.

The catheter 12 is a tube that can be inserted through an access port such as a portal or a duct and into a subcutaneous environment such as a body cavity duct or vessel and advanced to a wound site. The conduit 14 comprises flexible tubing sufficient for allowing the radioopaque zeolite to flow therethrough in the direction indicated by an arrow 18 under pressure from the positive pressure apparatus 16. Because the radioopaque zeolite (shown at 20) is preferably in particle or powder form, the inner surface of the conduit 14 is coated with or otherwise includes a suitable surface agent capable of facilitating the flow of the zeolite. One particular agent which the inner surface of the conduit 14 may be coated with is polytetrafluoroethylene (PTFE).

Once the radioopaque zeolite 20 is deposited at the wound site, the catheter 12 and the conduit 14 may or may not be retracted from the access port.

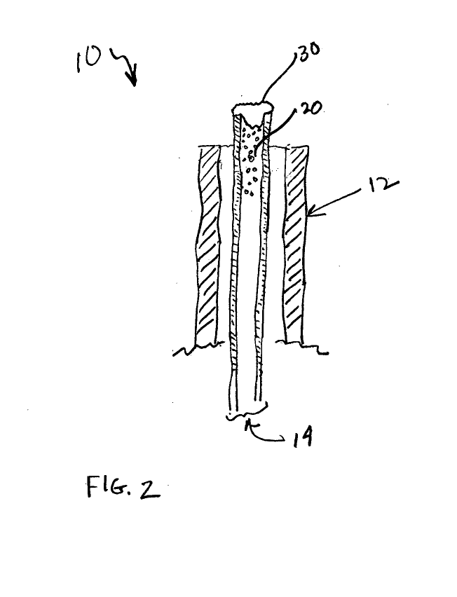

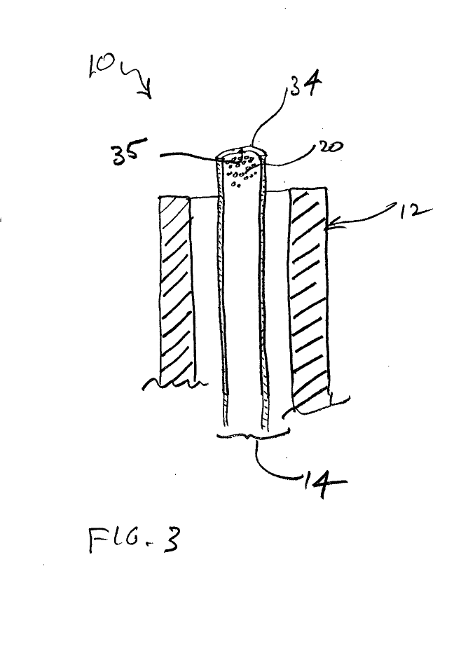

Referring now to FIGS. 2 and 3, the conduit 14 may be capped or otherwise prevented from receiving body fluids during the maneuvering of the device 10 in the subcutaneous environment. In FIG. 2, the conduit 14 may be capped using a wax plug or wax coating 30 to prevent the contact of the radioopaque zeolite 20 by both aqueous and non-aqueous body fluids. The wax coating 30 may be of a calculated thickness to allow the body heat of a patient into which the device 10 incorporating the wax coating 30 is inserted to melt the wax over a period of time. Preferably, the period of time is sufficient to allow the end of the conduit 14 to be positioned at the wound site without allowing body fluids to dissolve the wax. In FIG. 3, the conduit 14 may be capped with a thin membrane 34, which can be ruptured by pressurizing the conduit to a calculated pressure. In such an embodiment, the membrane 34 is preferably integrally formed with the conduit 14 and is sufficiently frangible to allow for rupture while not breaking off in the patient. An inner surface of the membrane 34 may include a notch 35 or similar defect to facilitate the rupture of the membrane. The membrane 34 is also sufficiently durable to prevent the penetration of the body fluids into the conduit 14, thereby preventing moisture accumulation by the zeolite 20.

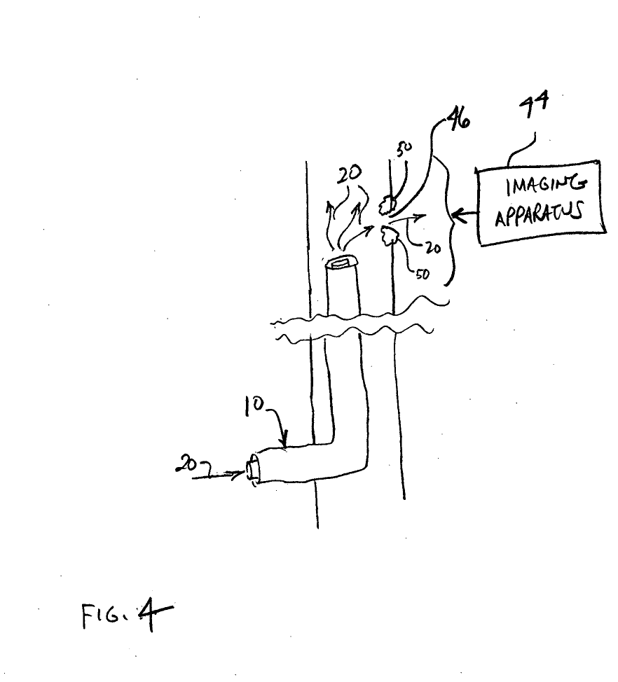

Referring now to FIG. 4, a system in which the radioopaque zeolite is imaged is shown generally at 40 and is hereinafter referred to as “system 40.” System 40 comprises the device 10 and an imaging apparatus 44. In using the system 40, the radioopaque zeolite 20 is deposited at or proximate the wound site (shown at 46) in a tubular organ 45 in which blood is present and the imaging apparatus 44 radiographically determines the flow of the zeolite as it is carried by the blood flow. As clots 50 form around or at the wound site 46, a series of images of the radiographic zeolite 20 taken over time will indicate a cessation of the blood flow from the high pressure side of the wound site to the low pressure side of the wound site. Although the device 10 of the system 40 is shown as being inserted into a tubular organ (e.g., a blood vessel or an esophagus) to stop the flow of blood from the high pressure side, it should be understood that the system may be utilized by inserting the device directly into the subcutaneous environment to address bleeding from the low pressure side.

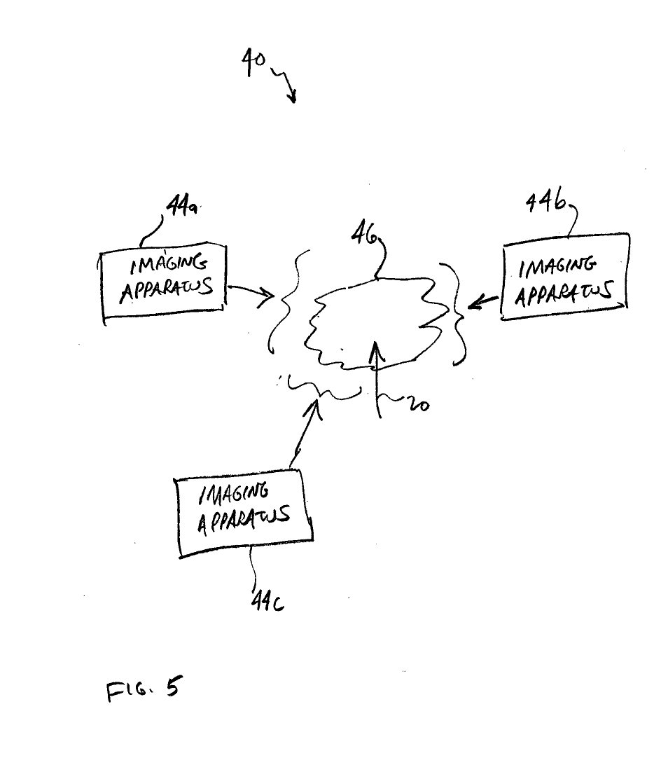

Referring now to FIG. 5, system 40 may incorporate a plurality of imaging apparatuses. One exemplary embodiment of system 40 comprises a first imaging apparatus 44a, a second imaging apparatus 44b, and a third imaging apparatus 44c. In utilizing three imaging apparatuses, three images may be radiographically obtained simultaneously. Using a suitable compilation technique, the radiographic data may be compiled to form a three-dimensional image of the wound site 46 for display on a suitable video apparatus. If a plurality of three-dimensional images is obtained over a period of time, the images may be sequentially viewed to illustrate the clotting of the blood over time.

As an alternative to compiling several images for three-dimensional viewing of the wound site 46, the first imaging apparatus 44a, the second imaging apparatus 44b, and the third imaging apparatus 44c may be operated in conjunction with a triangulation technique to accurately locate a wound site that can only be approximately located using cursory location methods. More specifically, standards can be developed (e.g., based on intensity of the imaged radioopaque zeolite) that enable distances to be calculated. By obtaining three or more values based on signal intensity from corresponding imaging apparatuses, a triangulation technique may be utilized to determine a conjunctive point that corresponds to a maximum intensity. Such a conjunctive point can be marked and subsequently determined to be a point of particular interest such as, for example, the point at which the radioopaque zeolite collects, which may be determined to be the point at which clotting is seeded.

In any embodiment in which zeolite is utilized as the blood clotting agent, the control of the moisture content of the zeolite in the substrate is related to its effectiveness. The preferred moisture content is between about 5 and about 25% by weight, more preferably between about 7 and about 19% by weight, and most preferably between about 10 and about 15% by weight. The moisture content of the zeolite can be adjusted by drying and then re-hydrating, or a combination of drying and re-hydrating, such that the zeolite has the desired specific moisture content. Alternatively, the composition may be fully saturated with water and subsequently dried to a specific water content. In the drying of the zeolite, the bound water is removed to allow the crystalline structure of the zeolite to remain intact. In the re-hydration of the zeolite, the most active adsorption sites are hydrated first and then less active sites are hydrated. As the degree of hydration of the zeolite increases, the heat of hydration decreases. More specifically, when the composition is applied to the blood, water in the blood is adsorbed by the zeolite. Upon adsorption of this water, heat is generated. At higher levels of hydration (hydration of the zeolite prior to its application to blood), less heat is generated when the composition is applied to blood. Thus, when the composition is applied to blood directly at a wound site, the amount of heat transferred to the tissue surrounding the wound site is reduced.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A radiographic composition for promoting the formation of clots in blood, said composition comprising:
a zeolite; and
a compound having an iodine ion associated with said zeolite;
wherein said iodine ion provides a radioopacity to said composition; and
wherein a moisture content of said composition is adjusted using a process selected from the group consisting of drying, re-hydrating, and a combination of drying and re-hydrating such that said composition has a specific moisture content.

2. The radiographic composition of claim 1, wherein said zeolite comprises aluminosilicate A-type crystals.

3. The radiographic composition of claim 1, wherein said zeolite is ion-exchanged to include a cation selected from the group consisting of calcium, sodium, potassium, silver, magnesium, and combinations of any of the foregoing cations.

4. The radiographic composition of claim 1, wherein said compound having said iodine ion is selected from the group consisting of iodipamide, iohexyl, compounds based on 5-amino-2,4,6-triiodo-N,N′-bis(2,3-dihydroxypropyl)isophthalamide, compounds based on 5-acetylamino-2,4,6-triiodo-N,N′-bis(2,3-dihydroxypropyl)isophthalamide, and combinations of the foregoing.

5. The radiographic composition of claim 1, wherein said compound having said iodine ion is bound to said zeolite.

6. The radiographic composition of claim 1, wherein said compound having said iodine ion is incorporated into a crystalline structure of said zeolite.

7. A device for promoting the clotting of blood at a bleeding internal wound site, the device comprising:
a catheter;
a delivery instrument controllably translatable through said catheter;
a radioopaque blood clotting agent deliverable through said delivery instrument, said radioopaque blood clotting agent comprising a molecular sieve material; and
a positive pressure apparatus capable of causing said radioopaque blood clotting agent to be delivered through said delivery instrument;
wherein when treating said bleeding wound site, depositing said radioopaque blood clotting agent at said bleeding wound site causes said molecular sieve material to come into contact with blood.

8. The device of claim 7, wherein said molecular sieve material is particulate in form.

9. The device of claim 7, wherein said molecular sieve material comprises,
a zeolite, and
a compound having an iodine ion associated with said zeolite,
wherein a moisture content of said molecular sieve material is adjusted to provide a desired exothermic effect at said internal wound site.

10. The device of claim 7, wherein said molecular sieve material is a zeolite.

11. The device of claim 10, wherein said zeolite further comprises a material selected from the group consisting of antibiotics, antifungal agents, antimicrobial agents, anti-inflammatory agents, analgesics, bacteriostatics, compounds containing silver ions, ascorbic acid, tranexamic acid, rutin, thrombin, botanical agents, and combinations of the foregoing.

12. The device of claim 10, wherein said zeolite comprises,
less than about 75% by weight of silicon oxide,
less than about 50% by weight of aluminum oxide,
less than about 30% by weight of sodium oxide, and
less than about 30% by weight of calcium oxide.

13. The device of claim 7, wherein said delivery instrument comprises a wax coating over an end thereof to prevent contact of said radioopaque blood clotting agent by body fluid.

14. The device of claim 7, wherein said delivery instrument comprises a frangible membrane over an end thereof to prevent contact of said radioopaque blood clotting agent by body fluid.

15. A system for radiographically imaging an internally bleeding wound, said system comprising:
means for delivering a radioopaque blood clotting agent to said internally bleeding wound; and
means for radiographically imaging said radioopaque blood clotting agent.

16. The system of claim 15, wherein said means for delivering said radioopaque blood clotting agent comprises,
a catheter, and
a delivery instrument controllably translatable through said catheter.

17. The system of claim 15, wherein said radioopaque blood clotting agent is a composition comprising,
a zeolite, and
a compound having an iodine ion associated with said zeolite,
wherein a moisture content of said composition is adjusted using a process selected from the group consisting of drying, re-hydrating, and a combination of drying and re-hydrating such that said composition has a specific moisture content.

18. A method of imaging an internally bleeding wound, said method comprising the steps of:
inserting a catheter into a subcutaneous environment;
advancing said catheter to a point adjacent said bleeding wound;
depositing a radioopaque zeolite from a delivery instrument in said catheter at said bleeding wound;
imaging said radioopaque zeolite; and
causing blood to clot at said bleeding wound.

19. The method of claim 18, wherein said step of causing blood to clot comprises contacting blood with said radioopaque zeolite.

20. The method of claim 18, wherein said zeolite comprises,
less than about 75% by weight of silicon oxide,
less than about 50% by weight of aluminum oxide,
less than about 30% by weight of sodium oxide,
less than about 30% by weight of calcium oxide, and
an effective amount of an iodine component.
